(19) United States
(12) Patent Application Publication
(10) Pub. No.: US 2010/0178292 A1
(43) Pub. Date: Jul. 15, 2010

(54) CRYPTIC GLYCAN MARKERS AND APPLICATIONS THEREOF

(75) Inventors: Denong Wang, Palo Alto, CA (US); Thomas Newsom-Davis, Twickenham (GB); Lawrence Steinman, Stanford, CA (US); Gavin Screaton, London (GB)

Correspondence Address: Stanford University, 1705 EL CAMINO REAL, PALO ALTO, CA, 94306, US

(73) Assignees: The Board of Trustees of the Leland Stanford Junior University, Palo Alto, CA (US); Imperial College of London, London (GB)

(21) Appl. No.: 12/686,102

(22) Filed: Jan. 12, 2010

Related U.S. Application Data
(60) Provisional application No. 61/144,143, filed on Jan. 12, 2009.

(51) Int. Cl. A61K 39/395; C07H 3/06; C07K 16/30; C07K 16/00; C07K 19/00; A61P 35/00; C12N 9/96; C12N 5/00; G01N 33/53; C40B 40/12; C40B 30/04

(52) U.S. Cl. 424/133.1; 536/123.1; 530/387.5; 530/387.3; 530/391.3; 424/137.1; 435/188; 435/325; 435/7.21; 506/19; 506/9

(57) ABSTRACT

The present invention relates to a novel glycan marker of cancer and monoclonal antibodies against it. Furthermore, novel glycan markers and their use in the detection and monitoring of cancerous cells and cancer-associated or specific antibody signatures are described.

### RELATED APPLICATION

This application claims priority and other benefits from U.S. Provisional Patent Application Ser. 61/144,143, filed Jan. 12, 2009, entitled “Cryptic Glycan Markers and Applications thereof”.

### STATEMENT OF GOVERNMENTAL SUPPORT

This invention was made with U.S. Government support under National Institutes of Health Grants RO1 NS055997 and UO1 CA128416. The Government has certain rights in this invention.

### TECHNICAL FIELD OF THE INVENTION

The present invention relates to the field of glycan biomarkers, anti-glycan antibodies and anti-glycan antibody signatures of certain cancers. It also relates to the use of antibodies which are directed against tumor-associated antigens for the detection of cancers, tumor imaging and the preparation of a pharmaceutical composition for the therapeutic intervention against cancer and application of glycan markers in tumor vaccination.

### BACKGROUND

Fas ligand (FasL) is a transmembrane protein that belongs to the tumor necrosis factor (TNF) family; FasL induces apoptosis in cells that express its receptor, Fas. Cell death mediated via Fas/FasL interaction is important for homeostasis of cells in the immune system and for maintaining immune-privileged sites in the body {Waring & Muellbacher (1999), Immunol. Cell Biol. 77 (4), pp. 312-317}. As a consequence, Fas ligand-receptor interactions play also an important role in the progression of cancer and, as such, are a primary target in cancer immunotherapy. When grafted into mice, tumor cells that are forced to overexpress FasL are rapidly rejected and an antibody-mediated tumor immunity develops {Seino et al. (1997), Nat Med 3, pp. 165-70; Shimizu et al. (1999), J Immunol 162, pp. 7350-7; Simon et al. (2002), Cancer Cell 2, pp. 315-22}.

The discovery of the hybridoma technology has made it possible to produce monoclonal antibodies of any desired specificity with relevance in many different therapeutic areas. For the use in cancer immunotherapy, monoclonal antibodies have been produced against a multitude of tumor-associated antigens. Tumor-associated antigens are structures which are expressed predominantly on the cell membrane of tumor cells and so allow differentiation from non-malignant tissues.

The antigenic determinants of a number of biologically important substances consist of carbohydrates; these often occur as glycoproteins or glycolipids. The predominant antigenic determinants of carbohydrate antigens may consist of either short oligosaccharides (one to six sugars long) at the nonreducing end of a sugar chain or as the internal chain structures of a polysaccharide {Wang D and Kabat E. A., Carbohydrate Antigens (Polysaccharides), (1996). Chapter 9, In: Structure of Antigens, Volume Three, (ed. M. H. V. Van Regenmortal), pp. 247-276, CRC Press, Boca Raton New York London Tokyo}. Approximately 80% of cell-surface proteins and 5% of lipids are glycosylated {Aarnoudse et al. (2006), Curr Opin Immunol 18, pp. 105-111}. Carbohydrate chains are typically displayed at the surface of cells and are potential antigens. Altered patterns of carbohydrate expression are one of the hallmarks of the cancerous cell, where changes include over- and underexpression of naturally occurring glycans, abnormal branching of glycoproteins and glycolipids, and neoexpression of sugars normally restricted to embryonic tissue {Hakomori (1989), Adv Cancer Res 52, pp. 257-331; Dube & Bertozzi (2005), Nat Rev Drug Discov 4, pp. 477-881. Abnormal glycosylation is associated with tumor cell invasion, metastasis and angiogenesis {Yoshimura et al. (1996), J Biol Chem 271, pp. 13811-5; Reddy & Kalraiya (2006), Biochim Biophys Acta 1760, pp. 1393-402}. For example, overbranching of N-linked glycans increases invasiveness, reduces contact inhibition, facilitates angiogenesis and promotes metastases by allowing tumor cell detachment from the tumor mass.

The importance of sugars as tumor antigens is now being realized, with over 50% of cancers being known to express the tumor-associated carbohydrate antigens (TACAs) described so far, including glycolipids (e.g. GM1, GM2, GD2 and GD3), Lewis antigens such as Le^{A}, Le^{X} and Le^{Y}, and Thomsen Friedenreich (TF) antigen {Slovin et al. (2005), Cancer Immunol Immunother 54 (7), pp. 694-7021.

Abnormal glycosylation on cancer cells makes carbohydrates attractive targets for cancer immunotherapy. The generation of an efficient adaptive immune response and, hence, the development of TACA vaccines has, however, been problematic due to the low immunogenicity of carbohydrate antigens, which, as self antigens, are typically not recognized as foreign. The assembly of multi-antigenic glycan vaccines, incorporation of carriers such as KLH, chemical modification of individual monosaccharides, and the use of endogenous adjuvants such as α-Gal antibodies have all been used with varying success to improve immunogenicity {Dube et al. (2005), Nat. Rev. Drug Discov. 4, pp. 477-488}.

FasL-expressing tumors have been described as a novel way to generate anti-carbohydrate antibodies that have the ability to confer immunity {Simon et al. (2008), Intl. Immunol. 20 (4), pp. 525-534.}

An ongoing challenge in cancer research is to identify reliable and accurate means to diagnose a tumor at the earliest stage possible. On the same note, it is important to develop diagnostic tools that help the physician to distinguish benign hypertrophied cells from malignant cells, which is still a challenge in cases such as prostate cancer. The availability of a “cancer-specific anti-glycan antibody signature profile” would be helpful in facilitating early diagnosis and early onset of proper treatment.

### SUMMARY

The following summary is not intended to include all features and aspects of the present invention nor does it imply that the invention must include all features and aspects discussed in this summary.

Embodiments of this invention describe a novel glyco-epitope that is aberrantly expressed by tumor cells, notably, its cell surface displays in a number of human cancers but not in corresponding normal cells, and that is defined by specific binding of monoclonal antibody TM10. In addition, monoclonal antibodies that specifically bind to this glyco-epitope as well as the respective hybridoma cell lines are provided. Furthermore, mannose-cluster arrays are described to display these antigenic determinants and detect antibodies against them. This technology can serve as diagnostic tool for the detection of antibody signatures of cancer in human serum and is potentially useful for the detection of cancers and for the monitoring of cancer progression. Further embodiments of the invention highlight the potential use of antibodies against glyco-epitopes in the development of therapeutic and/or prophylactic cancer vaccines.

### DRAWINGS

The accompanying drawings illustrate embodiments of the invention and, together with the description, serve to explain the invention. These drawings are offered by way of illustration and not by way of limitation.

FIG. 1 illustrates the generation of monoclonal antibodies from B16FasL vaccinated mice. mAbs were generated from ‘protected’ mice vaccinated with 1×10⁷ irradiated B16FasL cells and which had rejected at least three subsequent challenges with 5×10⁵ B16F10 cells. In total 5 monoclonal IgMs (TM3, TM5, TM6, TM10 and TM12) were produced all of which demonstrated cell surface binding to B16F10 (FIG. 1). Top row: B16F10 were stained with the mAbs indicated and analysed by flow cytometry. Open line—staining with mAb indicated. Bottom row: B16F10 (black line) and B16FasL (dashed line) were stained with the mAbs indicated. In all figures, closed line=isotype control staining of B16F10.

FIG. 2 illustrates that the generated monoclonal antibodies recognize both syngeneic and allogeneic murine tumors. The cell lines indicated were stained with the relevant mAb and analysed by flow cytometry. Closed line=isotype control, open line=mAb staining.

FIG. 3 illustrates that monoclonal antibodies TM 10 and TM12 recognize human cancer cells. Human melanoma (Trombelli, MM5), prostate (PC3, LN-CAP, DU145), ovarian (PEA-1, PEO-1, SK-OV-3) and breast (MDA-MB468, ZR75.1) cancer cells were stained with the relevant mAb and analysed by flow cytometry. Closed line=isotype control, open line=mAb staining.

FIG. 4 illustrates that monoclonal antibody TM 10 binds only to the surface of transformed cells. TM10 binds to the surface of tumor but not to untransformed cells, and there is a large intracellular reserve of its epitope in all cells. (A) Immunofluorescent microscopy (objective x63) of B16F10 cells surface stained and intracellular stained (permeabilised with Triton X-100) with TM10 or isotype control. Bar=10 μm. (B) Surface and intracellular (permeabilised with saponin) staining of human PBLs with TM10. Closed line—isotype control, open line=mAb staining.

FIG. 5 illustrates that monoclonal antibody TM 10 binds to a high mannose cluster epitope. (A) B16F10 cells were grown in the presence of (Top row) tunicamycin (inhibitor of N-linked glycosylation) for 18 hours, or (Bottom row) n-butyl-DNJ (inhibitor of α-glucosidase) for 72 hours. They were then stained with the mAbs indicated and analysed by flow cytometry. ConA—concanavalin A agglutinin. MAA—maakia amurensis agglutinin. Closed line=isotype control; black line=staining of untreated cells; dashed line=staining following incubation with relevant glycosylation inhibitor. (B) Carbohydrate microarray analysis of TM10 (10 μg/ml) showing specific binding to (Man9)n-KLH and [(Man9)4]ₙ-KLH, as compared to weak cross-reactivity to keyhole limpet hemocyanin (KLH) alone. (C) Graph of mean fluorescence intensity (MFI) of triplicate results from the array shown in B. Error bars represent SD.

FIG. 6 illustrates that the epitope that is recognized by monoclonal antibody TM 10 differs from the epitope that is recognized by lectins.

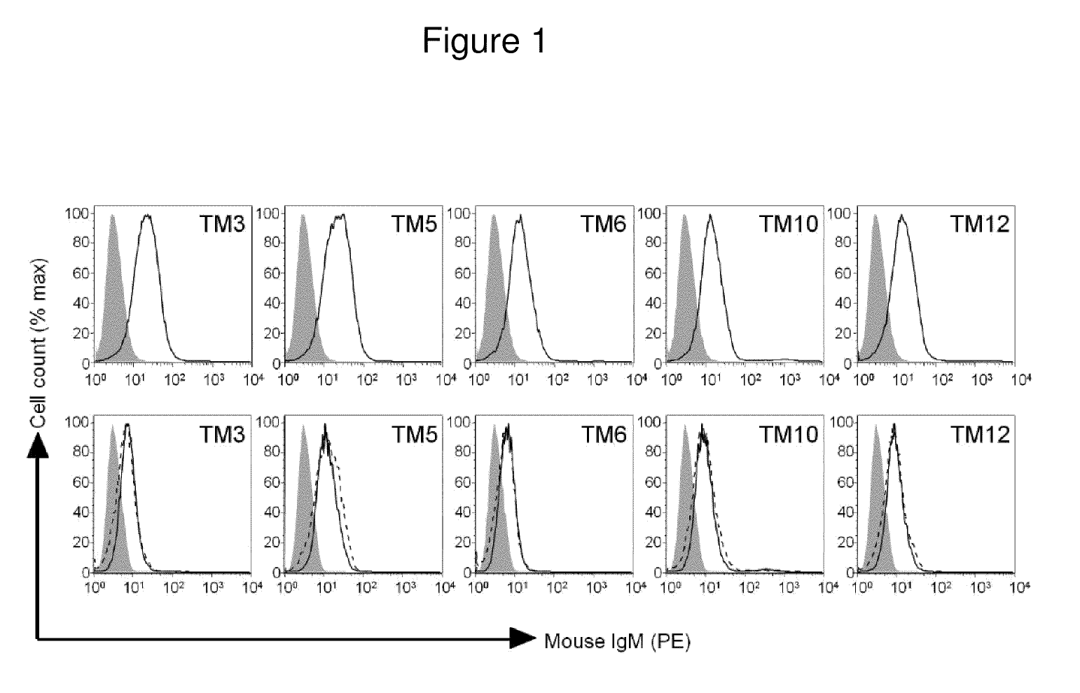

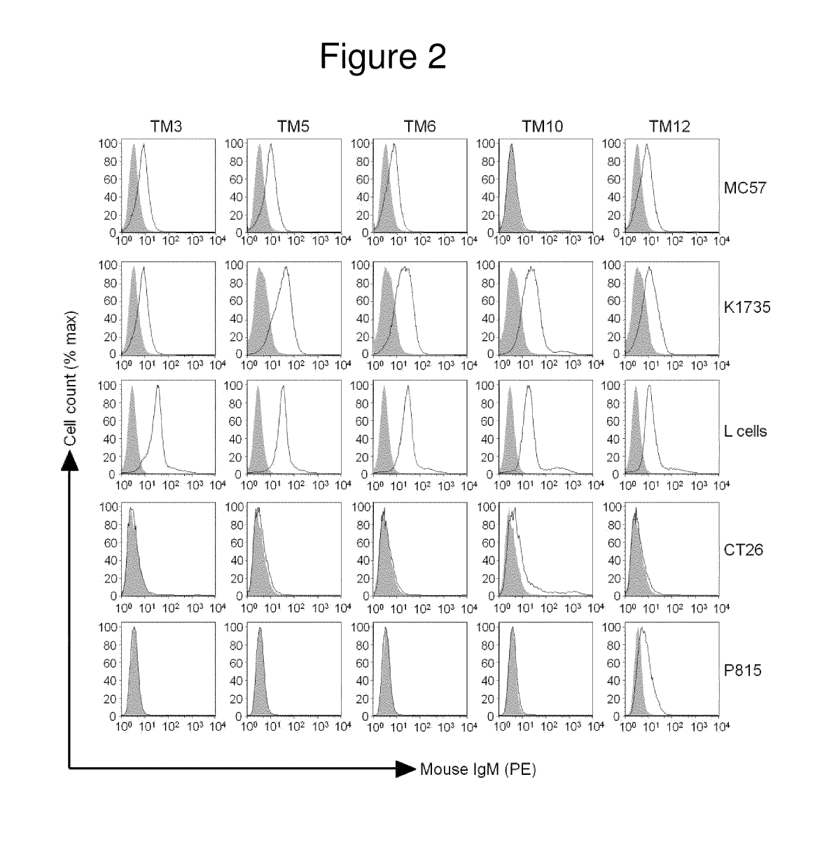

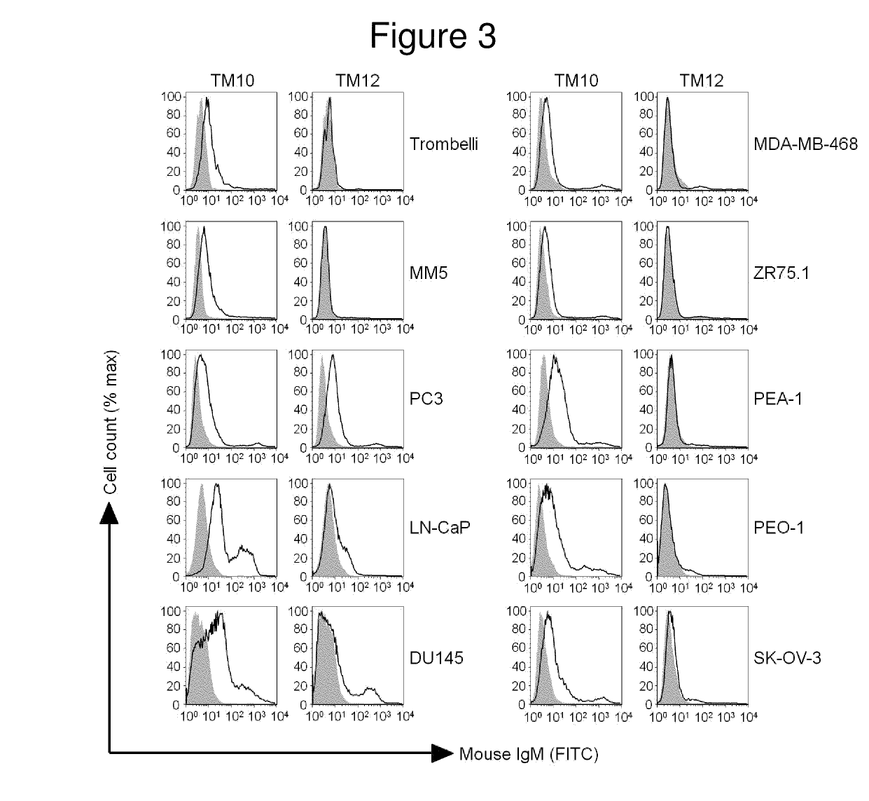

(A) 293T cells were cultured for 24 hours with or without 5 μM kifunensine and then stained with TM10 or MAA as indicated. Dashed line=plain culture medium; solid line=kifunsensine.
(B) B16F10 cells alone (left-hand graphs, solid line), or pre-incubated with SNA (top left graph, dashed line) or GNA (bottom left graph, dashed line), were stained with TM10. Alternatively B16F10 cells alone (right-hand graphs, solid line) or pre-incubated with TM10 (right-hand graphs, dashed line) were stained with SNA (top graphs) or GNA (bottom graphs). In all figures closed line=unstained B16F10 cells. MAA=maakia amurensis agglutinin, SNA=sambucus nigra agglutinin, GNA=galanthus nivalus agglutinin.

FIG. 7 shows Western blot and silver stain of immunoprecipitation with B16F10 and TM10 revealing multiple protein bands. B16F10 were lysed in NP40 lysis buffer and immunocprecipatated using protein L. (A) Western blot of 12% SDS-PAGE gel with anti-mouse IgM-HRP. (B) Silver stain of 12% SDS-PAGE gel. MW=molecular weight. Control=isotype control antibody.

FIG. 8 illustrates the N-glycosylation pathway in the endoplasmic reticulum (ER)/Golgi apparatus showing effects of glycosylation inhibitors (tunicamycin, N-butyl-DNJ, kifunensine). Asn=asparagine.

### DEFINITIONS

The practice of the present invention may employ conventional techniques of chemistry, molecular biology, recombinant DNA, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique’, Wiley-Liss (2000); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs.

The terms “glyco-epitope” and “carbohydrate epitope” are used interchangeably and characterize a region of a oligosaccharide or polysaccharide chain exhibiting a multitude of sugar residues on the surface of an antigen that is specifically or selectively recognized by an antibody, a lectin and/or other receptors.

The term “polysaccharide” refers to a polymeric sugar or so-called ‘glycan’, consisting of a plurality of monosaccharide residues that are joined with each other through glycosidic linkages. Polysaccharides with residues of less than 10 members are usually termed ‘oligosaccharides’ in the art.

The term “N-glycan” refers to an oligomeric or polymeric sugar chain that is attached to a protein or lipid through an asparagine-N-acetyl-D-glucosamine linkage. N-glycans can have different number of branches comprising various monosaccharides that are attached to the core structure.

The terms “mannose core”, “mannose core structure” or “core structure”, as used herein, refer to oligo- or polysaccharides which exhibit different sugar branches and which terminate in a number of mannose monosaccharide units. The two most prevalent mannose core structures herein are (Mang) which represents the mannose-core of N-glycoproteins and [(Man9)4]ₙ which mimics the mannose clusters displayed by the gp120 glycoprotein of HIV-1. Both mannose clusters were bound to the carrier keyhole limpet hemocyanin (KLH), but other carries may be used, as described (infra).

The term “biological sample” encompasses any sample consisting of or containing blood, serum, plasma, lymph fluid, amniotic fluid, saliva, cerebro-spinal fluid, lacrimal fluid, mucus, urine, sputum, or sweat.

The term “antibody” relates to antibodies of all possible types, in particular to monoclonal antibodies and also to antibodies produced by chemical, biochemical or genetic technological methods. The term “antibody” further includes various forms of modified or altered antibodies, such as various derivatives or fragments such as an Fv fragment, an Fv fragment containing only the light and heavy chain variable regions, an Fv fragment linked by a disulfide bond {Brinkmann, et al. Proc. Natl. Acad. Sci. USA, 90: 547-551 (1993)}, a Fab or (Fab)'2 fragment containing the variable regions and parts of the constant regions, a single-chain antibody and the like {Bird et al., Science 242: 424-426 (1998); Huston et al., Proc. Nat. Acad. Sci. USA 85: 5879-5883 (1988)}. The antibody may be of animal (especially mouse or rat) or human origin or may be chimeric {Morrison et al., Proc Nat. Acad. Sci. USA 81: 6851-6855 (1984)}. It may be humanized as described in Jones et al., Nature 321: 522-525 (1986), and published UK patent application #8707252.

The term “tumor-associated antigen” means a structure which is predominantly associated with tumor cells and thereby allows a differentiation from non-malignant cells.

The term “tumor-associated carbohydrate antigen” means a tumor-associated antigen that is a glycosylated protein, glycolipid or other carbohydrate-containing biological molecule that is associated with or specific for a tumor.

The term “carrier protein”, as used herein, means a protein suitable for conjugation to a high-mannose cluster including, but not limited to keyhole limpet hemocyanin (KLH), tetanus toxoid, diphtheria toxoid, bovine serum albumin and/or ovalbumin.

The term “passive vaccination” means evoking a specific immunity due to administration of antibodies against a tumor-associated antigen. In the context of the present invention, a vaccination can, in principle, be either carried out in a prophylactic or therapeutic fashion. A “prophylactic” vaccination is a vaccination administered to a mammalian subject in whom no cancerous cells were detected when the method to detect cancerous cells, as described in the present invention, was employed. A “therapeutic” vaccination is a vaccination administered to a mammalian subject in whom cancerous cells of breast, ovarian, prostate cancer or melanoma were detected when the method to detect cancerous cells, as described in the present invention, was employed.

Immunity, as used herein, means a specific host immune response that provides sufficient biological defenses to fight off a disease or pathological state temporarily or permanently.

The term “normal cell”, as used herein, characterizes a cell that exhibits regular cell division, while “abnormal”, as used herein, indicates unregular, but not yet uncontrolled cell division. The term “cancerous”, as used herein, characterizes a cell that exhibits uncontrolled cell division.

### DETAILED DESCRIPTION

Embodiments of the present invention describe mannose-cluster (Man9) based cryptic glycan markers that are highly and/or aberrantly expressed by numerous human cancers such as melanoma, prostate, ovarian and breast cancer and that can serve as novel glycan markers to detect transformed cells of those cancers. Although cryptic, i.e. masked by other sugar moieties, epitopes may not be exposed directly, they play an important role in the perpetuation of chronic inflammation through epitope spreading and such.

Further embodiments of the present invention describe the monoclonal antibody TM 10, which recognizes an epitope of the high-mannose clusters of N-linked glycans and which is able to specifically recognize transformed cells of human cancer lines such as melanoma, prostate, ovarian and, to less extent, breast cancer cell lines in addition to various syngeneic and allogeneic murine tumor cells lines. Monoclonal antibodies such as TM10 are instrumental in devising therapeutic and/or prophylactic cancer vaccines.

Other embodiments of the present invention describe a mannose-cluster array (glycan array) to detect anti-glycan antibodies such as monoclonal antibody TM 10 that specifically recognize carbohydrate epitopes of transformed cells as a means to detect and monitor immune response to malignant cell growth. The multitude of carbohydrates on the glycan array provides a cancer-specific anti-glycan antibody signature.

The detection of cancer-specific anti-glycan antibody signatures could provide diagnostic information to identify a subject as having or not having cancer, particularly with respect to melanoma, prostate cancer, and ovarian cancer.

The detection of cancer-specific anti-glycan antibody signatures will also provide valuable information for monitoring the progression or regression of cancer in a subject while receiving anti-cancer treatment.

Antibody Types, Fragments, Production and Detection

Antibodies which are also known as immunoglobulines (Ig) are, in their most abundant form of immunoglobulin G (IgG), usually heterotetrameric glycoproteins that are composed of two identical heavy and two identical light chains, whereby each light chain is linked to a heavy chain by one covalent disulfide bond. Each heavy chain has at one end a variable domain followed by a number of constant domains. Each light chain has a variable domain at one end and a constant domain at its other end. The constant domain of the light chain is aligned with the first constant domain of the heavy chain and the variable domain of the light chain is aligned with the variable domain of the heavy chain.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. Rather, it is concentrated in three segments called complementarity-determining regions (CDRs) or hypervariable regions {Wu, T. T. & Kabat, E. A. (1970), J. Exp. Med., 132, 211-250}. The more highly conserved portions of variable domains are called the framework. The CDRs in each chain are held together in close proximity by the framework regions and, with the CDRs from the other chain, contribute to the formation of the antigen-binding site of antibodies. Collectively, the six CDRs confer antigen-binding specificity to the antibody. However, even a single variable domain has the ability to recognize and bind antigen, although at a lower affinity than the entire binding site. {Wang D. and Kabat E. A. (1998). Antibodies, Specificity. In: Encyclopedia of Immunology, Second Edition, (ed. Delves and Roitt), Academic Press, London.; William Paul (2008), Fundamental Immunology, Sixth Edition, Lippincott William & Wilkins, Philadelphia}. The constant domains are not directly involved in binding an antibody to an antigen, but exhibit various effector functions.

Antibodies can be digested with the enzyme papain into two identical antigen-binding fragments called “Fab” fragments, each with a single antigen-binding site and a residual, readily crystallizable “Fc” fragment. Pepsin treatment yields an F(ab)₂ fragment that has two antigen-combining sites and is still capable of cross-linking antigen.

“Fv” is the minimum antibody fragment which contains a complete antigen-recognition and antigen-binding site. This region consists of a dimer of one heavy-chain and one light-chain variable domain in tight association.

Polyclonal antibodies are immunoglobulins of different specificities and originate from different B cells. They are a mixture of immunoglobulin molecules secreted against a specific antigen, each recognizing a different epitope. While polyclonal antibodies are easier to obtain, they are usually raised in animals such as rabbits and horses, they are likely specific for more than one epitope of an antigen.

Monoclonal antibodies, in contrast, are produced by the same B cell clone and are, therefore, identical copies of the same immunoglobulin; they are highly specific against a particular epitope. Monoclonal antibodies are typically made by fusing myeloma cells with the spleen cells from a mouse that has been immunized with the desired antigen (see infra and Simon AK (2002), Cancer Cell 2002; 2: 315-22).

Humanized antibodies or chimeric antibodies are types of monoclonal antibodies that have been synthesized using recombinant DNA technology to circumvent the clinical problem of immune response to foreign antigens. The standard procedure of producing monoclonal antibodies yields mouse antibodies. Although murine antibodies are very similar to human ones in their immunoglobulin structures, there are xenogenic to human hosts, and the human immune system recognizes mouse antibodies as foreign, rapidly removing them from circulation and causing systemic inflammatory effects.

Humanized antibodies are produced by merging the DNA that encodes the binding portion of a monoclonal mouse antibody with human antibody-producing DNA. One then uses mammalian cell cultures to express this DNA and produce these half-mouse and half-human antibodies that are not as immunogenic as the murine variety.

In certain embodiments, the antibody is immobilized on a solid phase, e.g. for diagnostic assays. For diagnostic uses, a labeled antibody (e.g. antibody bound to a detectable label) might be used; the labeling can be direct (i.e., physically linked) or indirect. Detectable labels can be fluorescerst, radioisotopes, enzymes, chemiluminescers or other labels for direct detection. Examples of indirect labeling include detection of a primary antibody using a fluorescently labeled secondary antibody and end-labeling a DNA probe with biotin such that it can be detected with fluorescently labeled streptavidin.

Antibody detection can be achieved using various methods, including flow cytometry, microscopy, radiography, scintillation counting, immunoassays.

Immunoglobulins. Immunoglobulins IgM and IgG are of particular importance for the present invention.

Immunoglobulin M or IgM, is a primary antibody isotype that is present on surfaces of B cells and produced by B cells. IgM antibodies are involved in the primary response upon the exposure to an antigen and appear early in the course of an infection and usually reappear, to a less extent, after further exposure. IgM also plays an important role in antibody-dependent cell-mediated cytotoxicity (ADCC).

Immunoglobulin G (IgG) is the most abundant immunoglobulin and synthesized and secreted by B cells. IgG antibodies are predominately involved in the secondary immune response. Only IgG can pass through the human placenta, thereby providing protection to the fetus in utero. IgG can bind to many different pathogens and protects the body against them by agglutination and immobilization, complement activation, phagocytosis and neutralization of their toxins. IgG also plays an important role in antibody-dependent cell-mediated cytotoxicity (ADCC).

### Therapeutic Use of Antibodies

Monoclonal antibodies that bind only to cancer cell-specific antigens and, as a consequence, induce an immunological response against certain targeted cancer cells have become a viable option in cancer therapy. Monoclonal antibodies, their fragments or derivatives, that are directed against tumor-associated antigens, can be used therapeutically or prophylactically in form of passive therapy or vaccination.

Therapeutic monoclonal antibodies can exert their anti-tumor effects through antibody-dependent cellular cytotoxicity and complement-dependent cytotoxicity with IgM antibodies being the most efficient isotype for complement activation {Adams & Weiner (2005), Nat Biotechnol 23, pp. 1147-1157}.

Direct therapeutic applications of monoclonal antibodies against a tumor-associated antigen are often based on the systemic administration of such antibodies, their fragments or their synthetic derivatives to cancer patients, once the presence of the particular antigen has been confirmed. The time course of the therapeutic effect typically correlates directly with the residence time and/or remaining concentration of such antibodies in the body; therefore, repeated or chronic administration is often necessary.

Another cancer immunotherapy approach is based on the selective activation of the immune system of cancer patients to combat and eliminate malignant cells before they can spread and cause metastases. Several different types of vaccinations are used for this purpose, including vaccinations with autologous or allogenic tumor cells with or without prior chemical or genetic modifications or vaccinations with isolated tumor-associated antigens, vaccinations with tumor-associated antigens which were produced by chemical or gene technological methods or vaccinations with peptide derivatives of such tumor-associated antigens or vaccinations with nuclear acids encoding for tumor-associated antigens.

The therapeutically effective immune response which is induced by vaccination with suitable antibodies against a tumor-associated antigen is determined by the binding region of these antibodies, i.e. by their idiotype. An alternative method of vaccination is based on the use of anti-idiotypic antibodies as an immunogenic substitute for a tumor antigen.

### Pharmaceutical Composition for Vaccination and Utility

In one embodiment, the present invention relates to a pharmaceutical composition for vaccinating a mammalian subject against cancer comprising either high-mannose clusters (active immunization) or at least one antibody such as monoclonal antibody TM10 that recognizes the described high-mannose clusters and high-mannose-carrier protein conjugates. (passive immunization). The pharmaceutical composition comprising either high-mannose clusters (active immunization) or at least one antibody in accordance to the use of the present invention may be administered as a vaccine with various pharmaceutically acceptable carriers that are commonly used in the formulation of vaccines. Pharmaceutically acceptable carriers include those approved for use in animals and humans and include but are not limited to diluents as well as adjuvants such as water, oils, saline, dextrose solutions, glycerol solutions and excipients such as starch, glucose, lactose, sucrose, gelatin, malt, rice, flour, chalk, silica gel, sodium stearate, glycerol monostearate, talc, sodium chloride, powdered non-fat milk, propylene glycol and ethanol. Pharmaceutical compositions may also include emulsifying agents or pH buffering compounds.

A composition of the present invention is typically administered parenterally in dosage unit formulations containing standard, well-known non-toxic physiologically acceptable carriers, adjuvants, and vehicles as desired. The term ‘parenteral’, as used herein, includes intravenous, intramuscular, intraarterial injection, or infusion techniques. Among the acceptable vehicles and solvents that may be employed are water, Ringer's solution, and isotonic sodim chloride (saline) solution. In addition, sterile oils are conventionally used as a solvent or suspending medium.

The compositions of the invention are administered in substantially non-toxic dosage concentrations sufficient to ensure the release of a sufficient dosage unit into a mammalian subject to provide the desired therapeutic immunity. The actual dosage administered and the frequency of dosage administration (vaccinating) will be determined by physical and physiological factors such as age, body weight, severity of condition and/or clinical history of the mammalian subject.

The therapeutic efficacy of the vaccination can be determined by the comparative detection of cancerous cells or a portion thereof in biological samples such as serum taken some time before and after the vaccinating step, using at least one antibody such as monoclonal antibody TM10 that recognizes the described high-mannose clusters and high-mannose-carrier protein conjugates. A decrease in detected cancerous cells or a portion thereof in biological samples such as serum taken after the vaccinating step, in comparison to biological samples taken before the vaccinating step, indicates therapeutic efficacy of the vaccination. Further vaccinating steps might be undertaken, as determined by the degree and sustainability of the efficacy of the vaccination.

### Detection of Cancerous Cells in Biological Samples

The presence of cancerous cells can be detected using antibodies against the cancer glycan markers.

### Carbohydrate Microarrays

In the field of glycomics research, carbohydrate microarrays are high-throughput discovery tools on a biochip platform which are useful for identifying immunologic sugar moieties, including complex carbohydrates of cancer cells and sugar signatures of microbial pathogens {Wang et al. (2002), Nature Biotechnology 21, pp. 275-281; Wang (2003), Proteomics 3, pp. 2167-2175; Wang, D. & Lu, J. (2004), Physiol. Genomics, 18, pp. 245-9; Wang et. al (2007), Proteomics 7, pp. 180-184.1. Within the field of immunology, carbohydrate microarrays are important tools to investigate the antigenic diversity of carbohydrate antigens. Carbohydrate microarrays can be designed as natural and/or synthetic mono-, di-, oligo- or polysaccharide chips as well as glycoconjugate chips.

### Mammalian Subjects

The above-described methods may be performed on a mammalian subject, e.g., a human, who is: a) not suspected of having a tumor, or b) suspected of having a tumor, to determine if that subject has a tumor, or c) known of having a particular tumor, to determine the progression or regression of that particular tumor.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, examples will be described to illustrate parts of the invention.

### EXAMPLES

### Example 1

### Only Tumor Cells, not Untransformed Cells Express the Carbohydrate Epitope that is Recognized by Monoclonal Antibody TM 10

Monoclonal antibody TM 10 binds only to the intracellular compartment of untransformed cells, not to the cell surface.

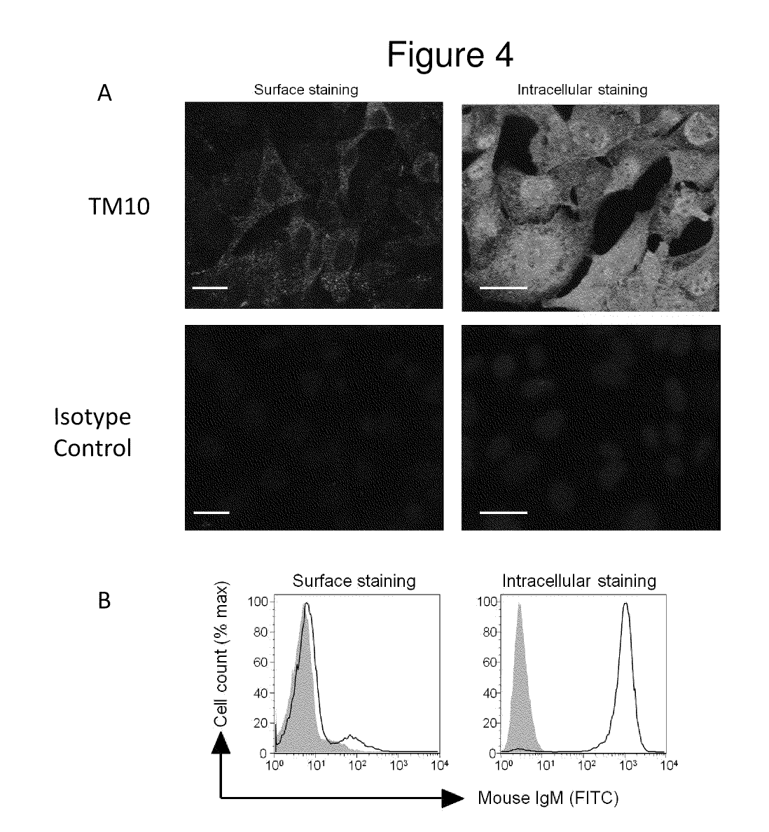

Using indirect immunofluorescence, TM10 was found to have a punctate surface staining pattern of B16F10 cells (FIG. 4A). Flow cytometry experiments did not show any cell surface binding of TM 10 to a range of untransformed cells including murine splenocytes and human PBLs (FIG. 4B), prostatic fibroblasts and dermal fibroblasts (data not shown). However, upon permeabilisation with Triton X-100, both normal and cancer cells showed strong intracellular staining (FIGS. 4A and 4B). This suggests that all cells have an intracellular reserve of the antibody's epitope, but it is only tumor cells that express it on their surface. There was no staining of any cells by isotype control antibody using direct immunofluorescence or flow cytometry.

### Example 2

### Monoclonal Antibody TM 10 Recognizes a Carbohydrate Epitope

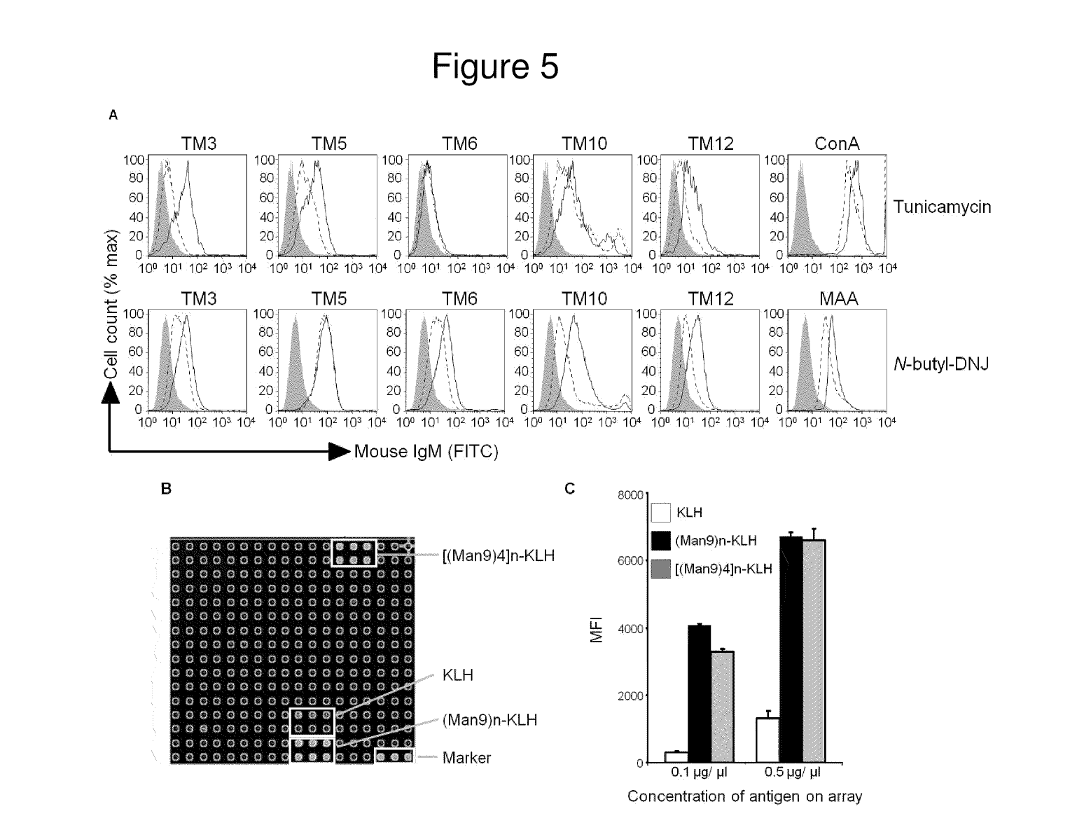

A protein epitope was initially sought. However, Western blots and immunoprecipitations from native, surface biotinylated or ³⁵S-methionine labelled B16F10 repeatedly revealed multiple protein bands (FIG. 7). This raised the possibility that our mAbs were in fact recognizing sugars expressed on more than one glycoprotein or glycolipid. Experiments using glycosylation inhibitors supported this. Tunicamycin, a mixture of homologous nucleoside antibiotics, is an inhibitor of N-glycoprotein synthesis (FIG. 8). When B16F10 cells were grown in the presence of tunicamycin, there was a reduction in staining by all the mAbs except for TM6 (FIG. 5A). The imino sugar N-butyl-deoxynojirimycin (N-butyl-DNJ) is a non-toxic inhibitor of α-glucosidase and prevents N-glycosylation one step downstream of the effects of tunicamycin (FIG. 8). There was reduced binding of all mAbs (except for TM5) when B16F10 cells were pre-treated with N-butyl-DNJ (FIG. 5A). As controls, tunicamycin also inhibited the binding of the lectin Con A that binds preferentially to mannose, and N-butyl-DNJ reduced binding of MAA, a lectin that preferentially recognises sialic acid which is one of the residues found on N-linked structures.

The carbohydrates recognized by the mAbs appeared to be restricted to glycoproteins and are not expressed on glycolipids. Incubation of B16F10 with another imino sugar, N-butyl-deoxy-galactonojirimycin (N-butyl-DGJ) which inhibits ceramide specific glucosyltransferase and so glycolipid but not glycoprotein formation, had no effect on mAbs binding (data not shown). Furthermore GM95, a B16F10 derived cell line that has reduced levels of ceramide specific glucosyltransferase and so impaired glycolipid expression, showed the same level of surface staining by all the mAbs when compared to B16F10 (data not shown).

### Example 3

### The Epitope for TM 10 is Displayed by High-Mannose Clusters (Man9)

With results pointing strongly towards carbohydrate epitopes, we screened our mAbs on a microarray of carbohydrates and glycolipids. TM10 bound strongly to two of these antigens (FIGS. 5B and 5C), both high-mannose clusters, displayed on the array as (Man9)ₙ—which represents the mannose-core of N-glycoproteins—and [(Man9)4]ₙ which mimics the mannose clusters displayed by the gp120 glycoprotein of HIV-1. Both mannose clusters were bound to the carrier keyhole limpet hemocyanin (KLH) but there was only weak binding of TM10 to KLH alone (FIG. 5B). Indeed, when the mean fluorescent intensities (MFIs) were compared at 0.1 μg/μl, (Man9)ₙ-KLH and [(Man9)4]ₙ-KLH gave a 14- and 11-fold increase in signal compared to that of KLH alone (FIG. 5C). It remains possible that the TM10 mAb may also have low affinity for some sugar epitopes on KLH. TM10 did not bind to any other antigens on the array, and there was no significant binding to the array of any of the other mAbs screened (data not shown).

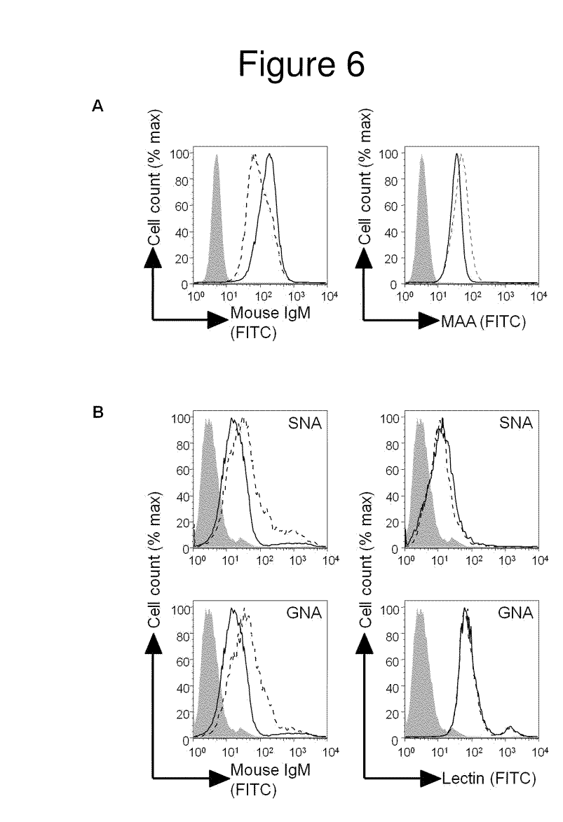

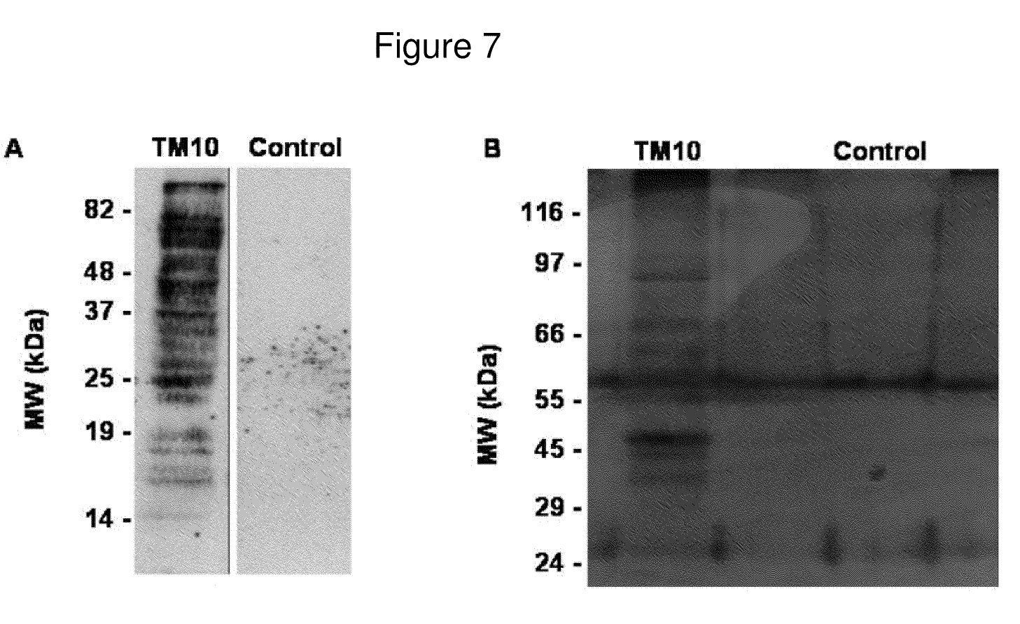

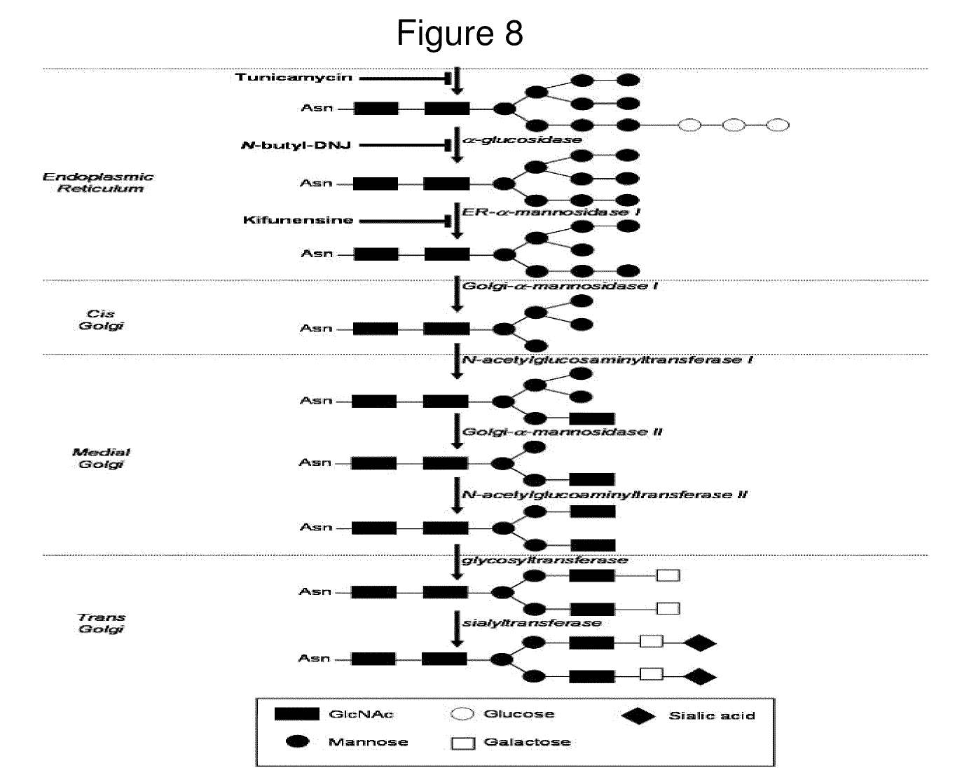

To confirm the array finding of a high-mannose cluster epitope for TM10, we used the α-mannosidase inhibitor kifunensine which prevents normal glycoprotein synthesis and leads to an accumulation of Man9 complexes (FIG. 8). Treatment of 293T cells with kifunensine increased the binding of TM10 confirming that the antibody is recognizing mannose clusters (FIG. 6A). As expected, the expression of sialic acid residues, detected through binding of MAA, was reduced in the presence of kifunensine. Inhibition experiments using D-(+)-mannose, D-(+) galactose, D-(+) glucose, D-(+) fucose, N-acetylglucosamine (GlcNAc), N-acetylgalactosamine (GluNAc) or mannose-6-phosphate to reduce binding of TM10 to B16F10 were negative (data not shown), suggesting that the antibody is recognizing a more complex structure than just these simple saccharide units.

Several different lectins known to bind to N-glycan complex carbohydrates were used to stain B16F10 and to competitively inhibit TM10 binding. SNA (binding preferentially to sialic acid attached to terminal galactose in (α-2,6) linkage), GNA (preferential binding to α1,3-mannose), and PHA-L (specific for Galβ1-4GlcNAc epitopes) all stained B16F10 (FIG. 6B and data not shown). However there was no inhibition of lectin binding when cells were pre-incubated with TM10. Furthermore, when B16F10 cells were pre-incubated with lectins there was no abrogation, but instead an increase, in TM10 binding. Taken together these findings suggest that the epitopes recognised by TM10 and the lectins differ, but that the target for TM10 may also be expressed on the lectins themselves. Lectins are glycoproteins, and SNA for example contains 7.8% carbohydrates, principally mannose and glucosamine.

### Example 4

### In-Vivo Anti-Tumor Effects of TM10 (IgM Isotype)

The in vivo anti-tumor effects of TM10 IgM were investigated but were disappointing as it did not significantly protect mice from the development of new melanomas nor retard the growth of existing tumors (data not shown). This can be explained, as IgM antibodies predominantly remain in the vasculature, have a shorter biological half-life and do not mediate antibody-dependent cellular cytotoxicity.

### (Prospective) Example 5

### In-Vivo Anti-Tumor Effects of Fv or Fab Domains of TM10 or Single Chain Antibodies with TM10 Binding Specificity (TM10 scFv)

To maximize the anti-tumor efficacy of the TM10 antibody, it is being cloned into a murine and human IgG isotype. The IgG class of antibodies are most efficient at mediating Fc domain based functions such as antibody dependent cellular cytotoxicity (ADCC).

The in vitro and in vivo anti-tumor effects of these antibodies will be determined.

### Experimental Procedures

### Cells Lines

Murine tumor cell lines used include B16F10 melanoma (syngeneic with the C57BL/6 mouse strain), K1735 melanoma (C3H), NS1 myeloma (BALB/c), MC57 fibrosarcoma (C57BL/6), CT26 colon carcinoma (BALB/c), methylcholanthrene (MCA)-transformed L-cell fibroblasts (C3H), P815 mastocytoma (DBA/2), and GM95 (ceramide specific glucosyltransferase deficient cells derived from B16F10 melanoma). FasL expressing B16F10 cells (B16FasL) were generated as described {Simon AK (2002), Cancer Cell 2002; 2: 315-22}.

Human cells used were primary prostate fibroblasts, dermal fibroblasts, 293T cells, melanoma (Trombelli, MM5), prostate (PC3, DU145, LN-CAP), breast (ZR75.1, MDA-MB 468), and ovarian cancer cell lines (PEA-1, PEO-1, SK-OV-3), kind gifts from Professor Jonathan Waxman, Dr Tahereh Kalamati, Professor Charles Coombes and Professor Hani Gabra, all of Imperial College London.

### Mice

Female C57BL/6 mice, aged 5-7 weeks, were purchased from Harlan (Oxon, UK) and housed at the Central Biomedical Services of Imperial College London. Non-hybridoma cells were cultured in RPMI 1640 medium supplemented with 10% heat-inactivated foetal calf serum (FCS), whilst hybridoma cells were supplemented with 20% batch-tested heat-inactivated FCS. Media were supplemented with 2 mM L-glutamine, 100 IU/m1 penicillin, 100 μg/ml streptomycin and, in the case of murine cells, 50 μM 2-mercaptoethanol, 10 mM HEPES, and 1% sodium pyruvate. All cells were incubated at 37° C. with 5% CO₂.

Generation and purification of monoclonal Antibodies

Murine hybridomas were generated by the fusion of splenocytes and myeloma cells as previously described {Simon AK (2002), Cancer Cell 2002; 2: 315-22}. Briefly, 1×10⁷ irradiated B16FasL melanoma cells were injected subcutaneously into female C57BL/6 mice which were then challenged three times, at monthly intervals, with 5×10⁵ B16F10 cells. Anti-tumor cell antibody production was confirmed in mice which rejected these tumor challenges (‘protected mice’) by staining of B16F10 cells with 1:50 diluted serum, the minimum concentration previously determined by titration to provide optimal staining. Splenocytes from protected mice with a positive anti-tumor cell antibody response were fused to NS1 murine myeloma cells using polyethylene glycol (PEG) 1500 (Roche Diagnostics, Basel, Switzerland). Hybridomas were selected by culture in HAT (hypoxanthine, aminopterine, thymidine) containing medium, and then screened by incubating their neat cell culture supernatant with B16F10 followed by anti-mouse Ig PE (Dako, Glostrup, Denmark). Positive staining hybridomas were single cell cloned three times and the class and subclass of each mAb determined using Isostrips (Roche).

Cell culture supernatants from hybridoma colonies were purified over a protein L column (Sigma-Aldrich, Dorset, UK), eluted with 0.1M glycine (pH 2.5) and dialysed in sterile PBS. mAb concentration was measured by spectrophotometer (280 nm optical density). mAbs were titrated and used at 10 μg/ml in the mircoarray and 20 μg/ml in all other experiments.

Flow Cytometry

Fc receptors on murine cells were blocked with rat anti-mouse CD16/32 (eBioscience, San Diego, Calif.) and human cells blocked with human serum (Gibco, Paisley, UK). In some experiments cells were fixed with 2% formaldehyde and then permeabilised using 0.5% saponin (Sigma-Aldrich). Secondary antibodies used were anti-mouse IgM FITC (Sigma-Aldrich) or anti-mouse Ig PE (Dako). A minimum of 2×10⁴ cells were analysed per sample.

Immunohistochemistry

B16F10 cells were grown to confluence on 15 mm glass coverslips and then fixed with 1% formaldehyde. Cells were blocked with 1:200 goat serum and, where indicated, permeabilised with 0.5% Triton X-100 (Sigma-Aldrich). They were incubated with mAbs (20 μg/m1) and then anti-mouse IgM Alexa Fluor-568 (Molecular Probes, Invitrogen, Paisley, UK) or anti-mouse IgM FITC. Cells were mounted onto glass slides with Vectashield/DAPI (Vector Laboratories, Peterborough, UK) and examined by confocal fluorescent microscopy using a x63 objective (Zeiss LS510, Jena, Germany).

Immunoprecipitation

1×10⁷ native or biotinylated (Biotin EZ-Link—Pierce, Cramlington, UK) B16F10 cells were lysed in 1 ml ice-cold NP40 lysis buffer and, for biotinylated samples, 0.5% Mega-9 (Sigma-Aldrich). Samples were pre-cleared with Protein L (Sigma-Aldrich), blocked with 10% bovine serum albumin (BSA), and then incubated with 20 μg/ml TM10 followed by 100 μL 50% protein L. The immunoprecipitates were run on 12% gels using SDS-PAGE and developed with silver staining (Amersham Biosciences, Little Chalfont, UK) or analysed by Western Blot.

Western Blot

1×10⁷ B16F10 cells were lysed in ice-cold NP40 lysis buffer, spun supernatants were separated on 12% gels using SDS-PAGE, and then transferred onto Hybond C Extra nitrocellulose membrane (Amersham). After being blocking with 5% non-fat dry milk/PBS, the membrane was incubated with 20 μg/m1 TM10, and then with anti-mouse Ig HRP (Sigma-Aldrich). HRP was detected using ECL Western Blotting kit (Amersham).

Glycosylation Inhibitors, Competitors, and Lectins

B16F10 cells were incubated at 37° C. for 24 hours with 200 ng/ml tunicamycin (Sigma-Aldrich) or for 72 hours with 1 mM N-butyl-deoxynojirimycin (N-butyl-DNJ) or N-butyl-deoxy-galactonojirimycin (N-butyl-DGJ) (Toronto Research Chemicals, Toronto, Canada). Alternatively 293T cells or peripheral blood lymphocytes (PBLs) were incubated overnight with 5 μM kifunensine (a gift from Dr Veronica Chang, Institute of Molecular Medicine, Oxford). Lectins, with or without conjugation to FITC, were derived from Sambucus Nigra (SNA), Maakia amurensis (MAA), Concanavalin A (ConA), Phaseolus vulgaris L (PHA-L), and Galanthus nivalis (GNA) (all Vector Laboratories, Burlingame, Calif.). D-(+)-mannose, D-(+)-galactose, D-(+)-glucose, N-acetylglucoasamine (GlcNAc), N-acetylgalactosamine (GluNAc) and mannose-6-phosphate (all Sigma-Aldrich) were used at 1 mg/ml. In some experiments pre-incubation with lectins or saccharides were used to inhibit binding of mAbs. In other experiments pre-incubation with the mAbs was used to inhibit the binding of the lectins or saccharides.

Carbohydrate Microarrays

Details of the protocol for the construction of carbohydrate microarrays have been previously published {Wang et al. (2005), Methods Mol Biol 310, pp. 241-52}. Briefly, carbohydrate and lipid antigens were printed in triplicate onto microglass slides using a robotic array spotter. Lipids were used at 20 μg/ml and carbohydrates at 0.5-1.0 μg/μ, initial concentration, and at a 1:5 dilution. Antibodies to murine IgM were also printed at given concentrations to serve as standard curves. The printed slides were blocked with BSA, incubated with the relevant mAb (10 μg/ml) and then anti-mouse IgG or IgM FITC. The analysis was performed using ScanArray Express Microarray Scanner (PerkinElmer Life Science, Boston, Mass.). Fluorescence intensity values for each spot were calculated with QuantArray software (Parkard Bioscience, PerkinElmer).

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

What is claimed is:

1. A glyco-epitope that is aberrantly expressed by tumor cells, including human melanoma (Trombelli, MM5), prostate (PC3, DU145, LN-CAP), breast (ZR75.1, MDA-MB 468), and ovarian cancer cell lines (PEA-1, PEO-1, SK-OV-3) and that is displayed by mannose-containing N-glycans of tumor cells and by high-mannose-cluster-carrier protein conjugates in carbohydrate microarrays.

2. An antibody that specifically binds to the glyco-epitope of claim 1.

3. The antibody of claim 2, wherein said antibody is a mouse monoclonal, mouse-human chimeric or humanized antibody, or a functional fragment thereof.

4. The antibody of claim 3, that is an IgM antibody.

5. The antibody of claim 3, that is an IgG antibody.

6. Monoclonal antibody TM10 that specifically binds to the glyco-epitope of claim 1 and that is produced by the hybridoma cell line TM10 and deposited with the ATCC on xx/xx/xxxx.

7. Functional fragments of the monoclonal antibody of claim 6, wherein the binding portion is selected from the group consisting of an Fab, an F(ab')₂ fragment and an Fv fragment.

8. The antibody of claim 2 wherein the antibody is conjugated to a label that produces a detectable signal.

9. The antibody of claim 8, wherein the label is selected from the group consisting of a radiolabel, an enzyme, a chromophore and a fluorescer.

10. Hybridoma cell line TM10, as deposited with the ATCC on xx/xx/xxxx.

11. A method to detect cancerous cells or a portion thereof in a biological sample comprising: providing an antibody or binding portion thereof which recognizes the glyco-epitope of claim 1, wherein the antibody is selected from claim 4, 5, 6 or 7, and wherein the antibody or binding portion thereof is bound to a label that allows detection of said cells.

12. The method of claim 11 wherein the tumor is melanoma.

13. The method of claim 11 wherein the tumor is prostate cancer.

14. The method of claim 11 wherein the tumor is breast cancer.

15. The method of claim 11 wherein the tumor is ovarian cancer.

16. A pharmaceutical composition for vaccinating a mammalian subject against cancer comprising at least one antibody directed against the glyco-epitope of claim 1.

17. The pharmaceutical composition of claim 16, wherein the antibody is selected from claim 4, 5, 6 or 7.

18. A method for inducing a therapeutic immunity by administering the pharmaceutical composition of claim 16 or 17 to a mammalian subject.

19. A carbohydrate microarray that displays a multitude of high mannose clusters and that can detect antibodies that specifically bind against these high mannose clusters to detect cancer autoantibody signature profiles so that a sample can be assessed as normal, abnormal or cancerous.

20. A method for detecting cancer autoantibody signature profiles in a biological sample according to claim 19, wherein the antibody is selected from claim 4, 5, 6 or 7.

21. A carbohydrate microarray that displays a multitude of structural and configural variants of synthetic high mannose clusters and that can detect antibodies that specifically bind against these high mannose clusters to detect cancer autoantibody signature profiles so that a sample can be assessed as normal, abnormal or cancerous.

22. A method for detecting cancer autoantibody signature profiles in a sample according to claim 21, wherein the antibody is selected from claim 4, 5, 6 or 7.
